(19) United States
(12) Patent Application Publication
(10) Pub. No.: US 2026/0131013 A1
(43) Pub. Date: May 14, 2026

(54) POLYMERIC COMPOUNDS FOR SURFACE MODIFICATION OF NATURAL KILLER CELLS TO INHIBIT METASTASIS OF PANCREATIC CANCER TO OTHER ORGANS

(71) Applicant: DONGGUK UNIVERSITY INDUSTRY-ACADEMIC COOPERATION FOUNDATION, Seoul (KR)

(72) Inventors: Kyobum Kim, Seoul (KR); Sungjun Kim, Seoul (KR)

(73) Assignee: DONGGUK UNIVERSITY INDUSTRY-ACADEMIC COOPERATION FOUNDATION, Seoul (KR)

(21) Appl. No.: 19/443,523

(22) Filed: Jan. 8, 2026

Related U.S. Application Data
(63) Continuation of application No. PCT/KR2024/011483, filed on Aug. 5, 2024.

(51) Int. Cl. A61K 47/60; A61P 35/04; C08B 37/08

(52) U.S. Cl. CPC A61K 47/60; A61P 35/04; C08B 37/0072

(57) ABSTRACT

Provided is a polymer compound capable of selectively recognizing pancreatic cancer cells and inhibiting metastasis of pancreatic cancer cells to other organs. The polymer compound may include a hydrophobic moiety binding to a natural killer (NK) cell, a cancer cell recognition moiety for selectively recognizing pancreatic cancer cells, and a linker for connecting the hydrophobic moiety and the cancer cell recognition moiety.

### CROSS-REFERENCE TO RELATED APPLICATIONS

This is a continuation of International Patent Application PCT/KR 2024/011483 (filed 5 Aug. 2024), the entire disclosure of which is hereby incorporated by reference herein.

### TECHNICAL FIELD

The present invention relates to a polymer compound which may be bound to a surface of a natural killer (NK) cell to enhance an anticancer function of the corresponding cell, in such a way that the polymer compound is synthesized, prepared, and injected to the body to selectively recognize and kill pancreatic cancer cells and inhibit metastasis of pancreatic cancer cells to other organs.

### BACKGROUND ART

Immunotherapy is one of the most successful and innovative methods for treating various cancers. Natural killer (NK) cells are special effect cells having a unique ability to identify and remove tumor cells without disadvantages of T cells such as a lack of autologous T cells, an off-target toxicity, and a cytokine release syndrome, and have recently attracted attention as cells capable of replacing T cells.

However, there is a problem in that an ability to selectively target tumor cells is insufficient due to an immunosuppressive tumor microenvironment (TME) and a lack of specific cancer-targeting ligands of NK cells, thus resulting in a decrease in an efficacy of immunotherapy.

Chimeric antigen receptor (CAR)-based genetic engineering is one of the technologies which may overcome a target limitation of NK cells and improve a therapeutic performance, and clinical trials are underway around the world. As interest and demand for CAR-NK cells increase, problems related to CAR-based engineering are also emerging.

Specifically, a process of preparing genetically engineered CAR-NK cells suffers from low infection efficiency, and a unique biological activity of NK cells for tumors may be reduced due to unpredictable mutagenesis. Accordingly, there is a demand for the development of new technologies which do not depend on genetic modification.

As an alternative to overcome limitations associated with a production of CAR-NK cells, surface engineering technology of NK cells is being studied. As a related technique, a technique of targeting CD22 by modifying a surface of NK cells through glycoengineering has been developed, but an expression of a target ligand through glycoengineering is entirely dependent on intracellular metabolism, and thus it is difficult to precisely control the target ligand.

### Technical Problem

One technical object of the present invention is to provide a polymer compound capable of binding to a cell membrane of NK cells. Another technical object of the present invention is to provide a polymer compound capable of selectively recognizing a pancreatic cancer cell to a surface of NK cells.

Still another technical object of the present invention is to provide a polymer compound capable of inhibiting metastasis of pancreatic cancer cells to other organs to a surface of NK cells.

Still another technical object of the present invention is to provide a polymer compound capable of modifying a surface of NK cells without genetic manipulation.

Still another technical object of the present invention is to provide a polymer compound which may be naturally removed from NK cells without an external physical and chemical intervention.

The technical objects of the present invention are not limited to the above-described objects.

### Technical Solution

To solve the technical problems described above, the present invention may provide a polymer compound.

According to one embodiment, there may be provided a polymer compound including a hydrophobic moiety binding to a natural killer (NK) cell, a cancer cell recognition moiety, and a linker, in which the hydrophobic moiety is bound to one end of the linker and the cancer cell recognition moiety is bound to the other end of the linker, so as to recognize NK cells and cancer cells, in which the cancer cell recognition moiety selectively recognizes pancreatic cancer cells, and a cell in which the NK cell and the polymer compound are bound inhibits metastasis of pancreatic cancer cells to other organs.

According to one embodiment, the cell in which the NK cell and the polymer compound are bound may inhibit metastasis of pancreatic cancer cells to lung.

According to one embodiment, the cancer cell recognition moiety may include hyaluronic acid.

According to one embodiment, the polymer compound bound to the NK cell through the hydrophobic moiety may bind to cancer cells through the cancer cell recognition moiety, and then promote a secretion of cytotoxic granules and cytokines from the NK cells to kill the cancer cells.

According to one embodiment, the hydrophobic moiety may include a lipid, and may be bound to a surface of the NK cell through a hydrophobic interaction via the lipid.

According to one embodiment, the hydrophobic moiety may include any one of a phospholipid having an alkyl chain having 12 to 24 carbon atoms, a sterols lipid having 10 to 30 carbon atoms, 1,2-distearoyl-sn-glycero-3-phosphorylethanolamine (DSPE), 1,2-bis(diphenylphosphino)ethane (DPPE), and 1,2-bis(dimethylphosphino)ethane (DMPE).

According to one embodiment, the linker may prevent the polymer compound bound to the NK cell from being subjected to endocytosis into the NK cell.

According to one embodiment, the linker may include polyethylene glycol (PEG).

According to one embodiment, the polymer compound bound to the NK cell may be removed from the NK cell within 36 hours.

According to one embodiment, the polymer compound bound to the NK cell may be naturally removed from the NK cell without an external physical and chemical intervention.

According to a third embodiment, the cancer cell recognition moiety may recognize solid cancer cells including CD44.

To solve the technical problems described above, the present invention may provide a method for preparing a polymer compound.

According to one embodiment, the method for preparing a polymer compound may include preparing hyaluronic acid, thiolating the hyaluronic acid, and reacting the thiolated hyaluronic acid with a compound in which a hydrophobic moiety is bound to one end of a linker and maleimide is bound to an other end thereof to prepare a polymer compound in which the hydrophobic moiety is bound to one end of the linker and the hyaluronic acid is bound to an other end thereof, in which pancreatic cancer cells are recognized by the hyaluronic acid, and NK cells are recognized by the hydrophobic moiety.

According to one embodiment, the polymer compound may be prepared by a Michael reaction between the thiolated hyaluronic acid and a compound in which a hydrophobic moiety is bound to one end thereof and maleimide is bound to an other end thereof.

According to one embodiment, the thiolating of the hyaluronic acid may include: conjugating the hyaluronic acid to 3-(2-pyridyldithithio)propionyl hydrazide (PDPH); and adding 2-mercaptoethanol to the hyaluronic acid to which the PDPH is conjugated.

### Advantageous Effects

A polymer compound including a hydrophobic moiety (lipid), a cancer cell recognition moiety (hyaluronic acid), and a linker (PEG) for connecting the hydrophobic moiety and the cancer cell recognition moiety may easily modify a surface of NK cells (modification such that the NK cell may recognize a specific cancer cell), and selectively recognize pancreatic cancer cells among various cancer cells. In addition, a cell in which the NK cell and the polymer compound are bound may inhibit metastasis of pancreatic cancer cells to other organs (lung).

### DESCRIPTION OF DRAWINGS

FIG. 1 is a view for describing a state in which a polymer compound according to an embodiment of the present invention is bound to an NK cell. FIGS. 2 and 3 are views for describing a cancer killing process through an NK cell to which a polymer compound according to an embodiment of the present invention is bound.

FIG. 4 is a view for describing a process in which a polymer compound according to an embodiment of the present invention is removed from an NK cell after being bound to the NK cell.

FIGS. 5 and 6 are views for describing a method for preparing a polymer compound according to an embodiment of the present invention.

FIG. 7 is a view comparing a HANK cell bound to AF-HA-SH and a HANK cell bound to AF-HA-PEG-Lipid.

FIGS. 8 and 9 are views for describing an effect of HA-PEG-Lipid on NK cells.

FIGS. 10 and 11 are views for confirming a target recognition ability of a HANK cell.

FIG. 12 is a graph comparing an amount of cytotoxic granules and cytokines secreted between NK cell and HANK cell for target cells.

FIG. 13 is a graph comparing a lysis ability between NK cell and HANK cell for cancer cells.

FIG. 14 and FIG. 15 are views comparing an anticancer efficacy between NK cell and HANK cell for tumor spheroids.

FIG. 16 is a view for confirming a retention time of HA-PEG-Lipid bound to NK cells.

FIG. 17 is a view comparing a cytokine secretion ability between NK cell and a restored NK cell.

FIG. 18 is a view comparing a cancer cell lysis ability between NK cell and a restored NK cell.

FIG. 19 is a view showing an experimental process for confirming an anticancer efficacy of HANK cell for pancreatic cancer.

FIGS. 20 and 21 are views showing a change in volume, weight, and size of a tumor according to a progress of an experiment on pancreatic cancer.

FIG. 22 is a view comparing a tumor penetration ability between NK cell and HANK cell.

FIG. 23 is a view for confirming a biodistribution of NK cell and HANK cell injected into experimental mice.

FIG. 24 and FIG. 25 are view comparing a cytotoxic granule spreading ability between NK cell and HANK cell.

FIG. 26 and FIG. 27 are views comparing a cytokine spreading ability between NK cell and HANK cell.

FIG. 28 and FIG. 29 are views comparing a tumor necrosis ability among PBS, NK cell, gemcitabine, and HANK cell.

FIG. 30 and FIG. 31 are views comparing an apoptotic region of tumor masses via PBS, NK cell, gemcitabine, and HANK cell.

FIG. 32 and FIG. 33 are view comparing a cell proliferation inhibitory ability among PBS, NK cell, gemcitabine, and HANK cell.

FIG. 34 is a view showing an experimental process for evaluating an antimetastatic effect of HANK cell.

FIG. 35 is a view confirming a metastatic colony formation in

lung tissues.

FIG. 36 is a view for confirming an alveolar structure in lung tissues.

FIG. 37 is a view for describing the result of introducing a human leukocyte antigen into lung tissues.

FIG. 38 is a view for describing the result of introducing an angiogenesis marker into lung tissues.

FIG. 39 is a view quantifying the number of metastatic foci in the lung identified in the lung of experimental mice injected with PBS, NK cell, gemcitabine, and HANK cell.

FIG. 40 is a view quantifying an hLA positive cell area identified in the lung of experimental mice injected with PBS, NK cell, gemcitabine, and HANK cell.

FIG. 41 is a view quantifying a vWF positive cell area identified in the lung of experimental mice injected with PBS, NK cell, gemcitabine, and HANK cell.

### MODE FOR INVENTION

Hereinafter, preferred embodiments of the present invention will be described in detail with reference to the accompanying drawings. However, the technical spirit of the present invention is not limited to the embodiments described herein and may be implemented in other forms. Rather, the embodiments introduced herein are provided so that the disclosed contents may be thorough and complete and the spirit of the present invention may be sufficiently conveyed to those skilled in the art.

In this specification, when a component is referred to as being on another component, it means that it may be formed directly on the other component or a third component may be interposed therebetween. In addition, in the drawings, the thicknesses of films and regions are exaggerated for effective description of the technical contents.

Furthermore, in various embodiments of the present specification, terms such as first, second, third, etc., are used to describe various components, but these components should not be limited by these terms. These terms have only been used to distinguish one component from another component. Accordingly, a component mentioned as a first component in one embodiment may be mentioned as a second component in another embodiment. Each embodiment described and exemplified herein includes a complementary embodiment thereof. In addition, in the present specification, “and/or” is used as a meaning including at least one of the components listed before and after.

In the specification, a singular expression includes a plural expression unless the context clearly indicates otherwise. In addition, terms such as “include,” “have” or the like are intended to designate the presence of features, numbers, steps, components, or combinations thereof described in the specification, and should not be understood to preclude the possibility of the presence or addition of one or more other features, numbers, steps, components, or combinations thereof. In addition, in the present specification, “connection” is used as a meaning including both indirectly connecting a plurality of components and directly connecting the plurality of components.

Furthermore, in the following description of the present invention, when it is determined that a detailed description of a related known function or configuration may unnecessarily obscure the subject matter of the present invention, the detailed description thereof will be omitted.

Polymer compound according to embodiment

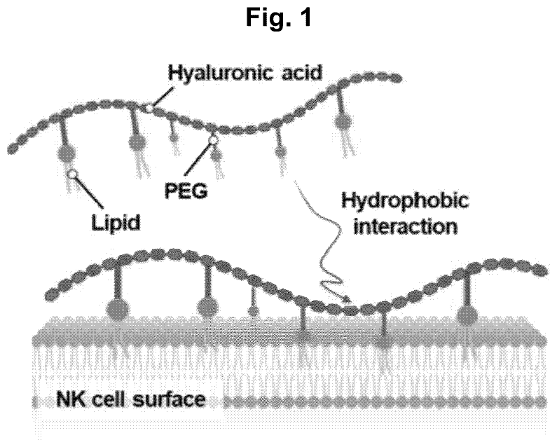

FIG. 1 is a view for describing a state in which a polymer

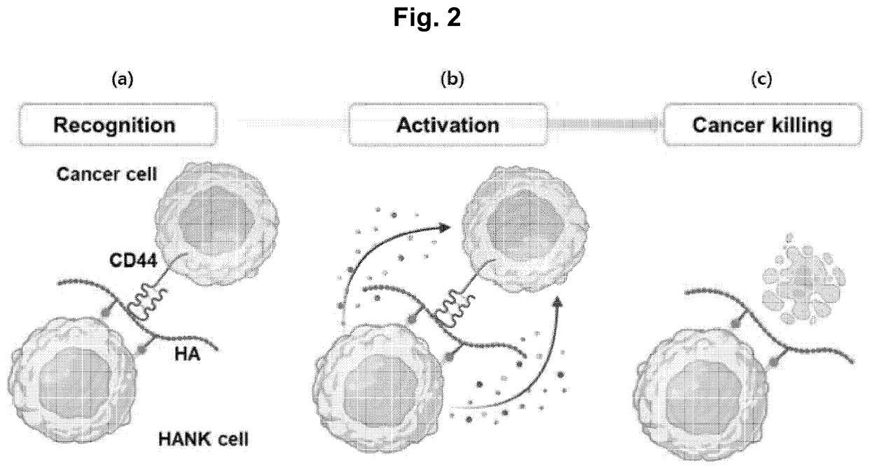

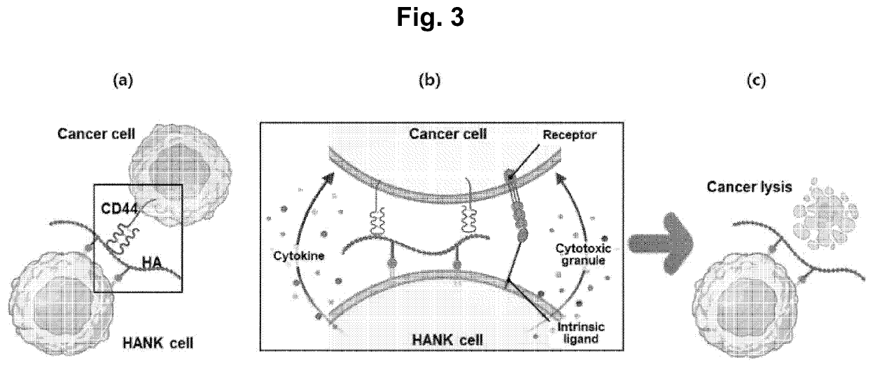

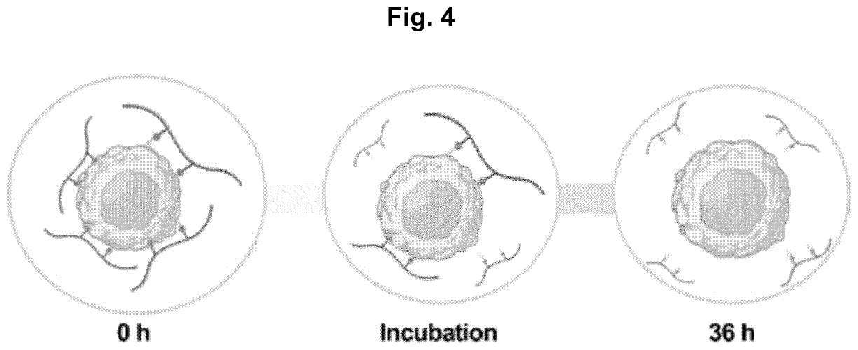

compound according to an embodiment of the present invention is bound to an NK cell, FIGS. 2 and 3 are views for describing a cancer killing process through an NK cell to which a polymer compound according to an embodiment of the present invention is bound, and FIG. 4 is a view for describing a process in which a polymer compound according to an embodiment of the present invention is removed from an NK cell after being bound to the NK cell.

Referring to FIG. 1, the polymer compound according to an embodiment of the present invention may include a hydrophobic moiety, a cancer cell recognition moiety, and a linker for connecting the hydrophobic moiety and the cancer cell recognition moiety. In other words, the polymer compound may have a structure

in which the hydrophobic moiety is bound to one end of the linker and the cancer cell recognition moiety is bound to the other end thereof.

The hydrophobic moiety may recognize a natural killer (NK) cell and may be bound to a surface of the NK cell. Accordingly, the polymer compound may be immobilized on the surface of the NK cell by the hydrophobic moiety. More specifically, the hydrophobic moiety may be configured to include a lipid, and may be bound to the surface of the NK cell through a hydrophobic interaction via the lipid.

According to one embodiment, the hydrophobic moiety may include any one of a phospholipid having an alkyl chain having 12to 24 carbon atoms, a sterols lipid having 10 to 30 carbon atoms, 1,2-distearoyl-sn-glycero-3-phosphorylethanolamine (DSPE), 1,2-bis(diphenylphosphino)ethane (DPPE), and 1,2-bis(dimethylphosphino)ethane (DMPE).

The cancer cell recognition moiety may recognize a cancer cell and bind to the cancer cell, and may include hyaluronic acid. As described above, when the cancer cell recognition moiety includes hyaluronic acid, the cancer cell recognition moiety may selectively recognize solid cancer cells including CD44. More specifically, the cancer cell recognition moiety including hyaluronic acid may selectively recognize cancer cells with CD44overexpressed thereon. For example, cancer cells with CD44overexpressed thereon may include pancreatic cancer cells (MIA PaCa-2), triple negative breast cancer cells (MDA-MB-231), and colorectal cancer cells (HCT-116). In other words, the cancer cell recognition moiety including hyaluronic acid may selectively recognize pancreatic cancer cells (MIA PaCa-2), triple negative breast cancer cells (MDA-MB-231), and colorectal cancer cells (HCT-116) among various cancer cells. Accordingly, the NK cell bound to the hydrophobic moiety may selectively kill pancreatic cancer cells (MIA PaCa-2), triple negative breast cancer cells (MDA-MB-231), and colorectal cancer cells (HCT-116) among various cancer cells by the cancer cell recognition moiety.

The linker may be adapted for connecting the hydrophobic moiety and the cancer cell recognition moiety, and may include polyethylene glycol (PEG). In addition, the linker may prevent the polymer compound bound to the NK cell from being subjected to endocytosis into the NK cell.

Referring to FIGS. 2 and 3, a process in which the polymer compound bound to the NK cell through the hydrophobic moiety kills cancer cells may be shown. In FIGS. 2 and 3, the cell in which the polymer compound and the NK cell are bound may be represented by HANK cell. In addition, (a) of FIGS. 2 and 3 may show the recognizing of HANK cell, (b) may show the activating, and (c) may show the killing of cancer cells.

Specifically, the HANK cell may recognize cancer cells including CD44 through the cancer cell recognition moiety (hyaluronic acid, HA) of the polymer compound, and then may be bound to cancer cells through the cancer cell recognition moiety (hyaluronic acid, HA). After that, an activation process in which cytotoxic granules and cytokines are secreted from the NK cell may be performed, and cancer cells may be killed by the cytotoxic granules and cytokines secreted from the NK cell. According to one embodiment, the polymer compound may promote the secretion of cytotoxic granules and cytokines from the NK cell during the activation process, thereby improving cancer cell killing efficiency.

Referring to FIG. 4, the polymer compound bound to the NK cells may be naturally removed from the NK cell within 36 hours without an external physical and chemical intervention. Unlike the above description, when the polymer compound bound to the NK cell is not removed, various problems such as cytokine release syndrome, neurotoxicity, off-tumor effects, and acute respiratory distress syndrome may occur.

In order to solve these problems, a method of introducing and removing a suicide gene has been conventionally used, but another problem has occurred because additional drug treatment for activating the suicide gene needs to be performed. However, since the polymer compound may be naturally removed from the NK cell without an external physical and chemical intervention, the above-described problems may be easily solved.

In addition, the binding and removal of the polymer compound for the NK cell may not impair an intrinsic function of the NK cells. In other words, even after the polymer compound is removed from the NK cell, the NK cell may maintain an inherent function thereof.

As a result, a polymer compound including a hydrophobic moiety (lipid), a cancer cell recognition moiety (hyaluronic acid), and a linker (PEG) for connecting the hydrophobic moiety and the cancer cell recognition moiety may easily modify a surface of NK cells by a hydrophobic interaction through the hydrophobic moiety (lipid) without genetic manipulation (modification such that the NK cell may recognize a specific cancer cell), and may selectively recognize solid cancer cells (e.g., pancreatic cancer cells, triple negative breast cancer cells, colorectal cancer cells, etc.) with CD44 overexpressed thereon among various cancer cells. In addition, the polymer compound may be naturally removed without an external physical and chemical intervention within a predetermined time (within 36 hours), and thus problems caused by a long-term surface modification of NK cells (e.g., cytokine release syndrome, neurotoxicity, off-tumor effects, acute respiratory distress syndrome, etc.) may be easily solved.

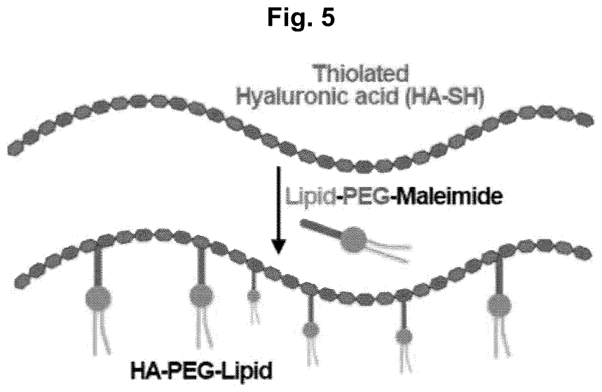

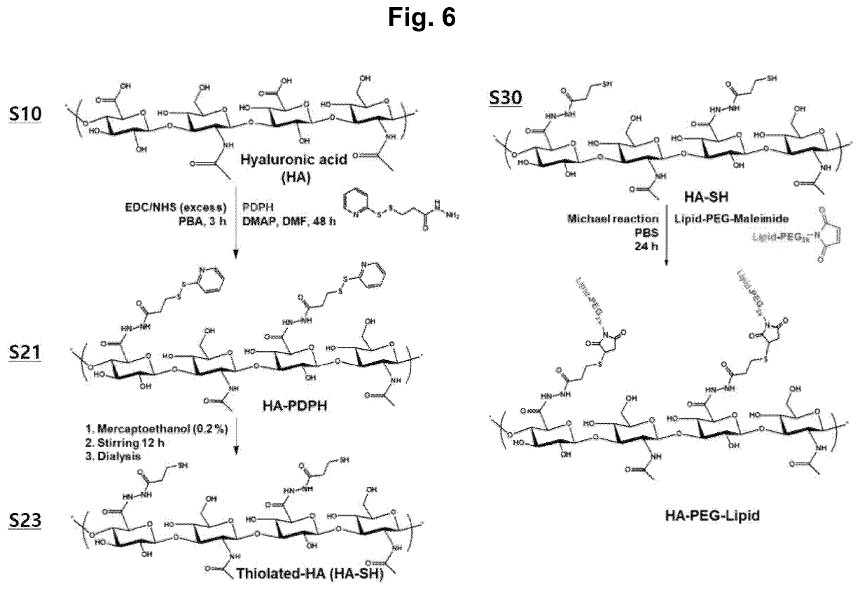

Method for preparing polymer compound according to embodiment FIGS. 5 and 6 are views for describing a method for preparing a polymer compound according to an embodiment of the present invention.

Referring to FIGS. 5 and 6, the method for preparing a polymer compound according to an embodiment of the present invention may include preparing hyaluronic acid (S10), thiolating the hyaluronic acid (S21, S22), and preparing the polymer compound by using the thiolated hyaluronic acid (S30). Hereinafter, each step will be described in detail. In above S10, hyaluronic acid (HA) may be prepared. According to one embodiment, in above S10, 1-ethyl-3-(3-dimethylaminopropyl) carbodiimide (EDC) and N-hydroxy succinimide (NHS) may be added to a mixed solution of a hyaluronic acid solution and phosphate-buffered saline (PBS) to activate a carboxyl group.

In above S 21, the hyaluronic acid may be conjugated with 3-(2-pyridyldithiothio)propionyl hydrazide (PDPH). According to one embodiment, in above S21, the hyaluronic acid prepared in above S10 may be mixed with 3-(2-pyridyldithithio)propionyl hydrazide (PDPH), 4-dimethyl amino pyridine (DMAP), and dimethyl formamide (DMF) to conjugate the hyaluronic acid with the PDPH. The compound in which the hyaluronic acid is conjugated with PDPH may be defined as HA-PDPH.

In above S 22, 2-mercaptoethanol may be added to the hyaluronic acid with which the PDPH is conjugated (HA-PDPH) to prepare a thiolated hyaluronic acid (thiolated-HA, HA-SA).

Finally, in above S30, the thiolated hyaluronic acid (HA-SH) may be subjected to a Michael reaction with a compound in which a hydrophobic moiety (lipid) is bound to one end of the linker and maleimide is bound to the other end thereof. Accordingly, the polymer compound (HA-PEG-Lipid) in which the hydrophobic moiety (lipid) is bound to one end of the linker (PEG) and the hyaluronic acid (HA) is bound to the other end thereof may be prepared.

Hereinafter, the polymer compound according to an embodiment of the present invention will be described in more detail through specific experimental examples.

Preparation of polymer compound (HA-PEG-Lipid) according to experimental example

A hyaluronic acid (HA) solution (Mw 60 k, 10 mg/mL, 1 equiv., LifeCore Biomedical) and phosphate-buffered saline (PBS, pH 7.4) were stirred for 30 minutes, after which an excessive amount of 1-ethyl-3-(3-dimethylaminopropyl) carbodiimide (EDC, Sigma-Aldrich) and N-hydroxy succinimide (NHS, Sigma-Aldrich) were added to the stirred solution and reacted at room temperature for three hours to activate a carboxyl group.

After that, 3-(2-pyridyldithio)propionyl hydrazide (PDPH, Sigma-Aldrich) (30 equiv.), 2 mg of 4- dimethyl amino pyridine (DMAP, Sigma-Aldrich), and dimethyl formamide (DMF, Sigma-Aldrich) were further added thereto and reacted at room temperature for 72 hours to obtain a HA-PDPH product in which PDPH was conjugated to HA. In addition, the resulting HA-PDPH product was dialyzed (MWCO 2 kDa) with distilled water for three days and lyophilized to remove unconjugated PDPH and EDC/NHS therefrom.

After 30 mg of HA-PDPH was dissolved in 10 mL of PBS, 2-mercaptoethanol was added at an initial concentration of 0.2 wt % and stirred at room temperature for 12 hours. A mixture resulting from stirring was dialyzed (MWCO 2 kDa) with distilled water for three days and then lyophilized to prepare a thiolated HA (HA-SH).

50 mg of HA-SH was dissolved in 10 mL of PBS to prepare a homogeneous HA-SH solution, and then a solution in which Lipid-PEG-Maleimide (30 eq.) was dissolved in 10 mL of DMF was added to the resulting solution, and stirred at room temperature for 24 hours. The stirred mixture was dialyzed (MWCO 12-14 kD) with distilled water for three days and lyophilized to prepare HA-PEG-Lipid as a final product. More specifically, 1,2-distearoyl-sn-glycero-3-phosphatidylethanolamine (DSPE) was used as the lipid. In addition, the preparation method described above is shown in FIG. 6.

### Preparation of HANK Cell According to Experimental Example

The polymer compound (HA-PEG-Lipid) according to above experimental example was dissolved in MEM alpha, and 5×10⁵ NK- 92 mi NK cells were evenly mixed in 100 μL of a solution, and then reacted at room temperature for 30 minutes to prepare HANK cells in which the polymer compound (HA-PEG-Lipid) according to above experimental example was bound to NK cells.

### Experimental Example 1: Analysis of HA-PEG-Lipid and HANK Cell Properties

20 mg of HA-PEG-Lipid was dissolved in 2 mL of PBS and 100 nmol of Alex Flour 488 hydrazide (fluorescent dye) was added to prepare AF-HA-PEG-Lipid. In addition, 20 mg of HA-SH was dissolved in 2 mL of PBS and 100 nmol of Alex Flour 488 hydrazide (fluorescent dye) was added to prepare AF-HA-SH. After that, AF-HA-PEG-Lipid and AF-HA-SH were bound to NK cells to prepare HANK cells.

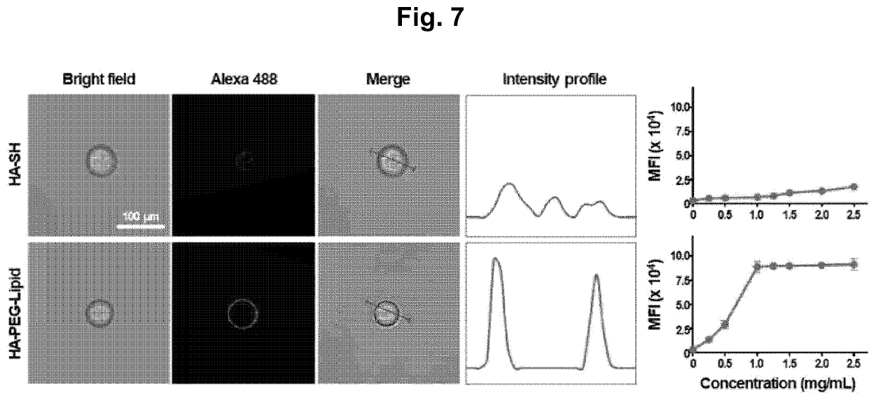

FIG. 7 is a view comparing a HANK cell bound to AF-HA-SH and a HANK cell bound to AF-HA-PEG-Lipid.

Referring to FIG. 7, it may show the optical and fluorescence microscopy images of HANK cell bound to AF-HA-SH and HANK cell bound to AF-HA-PEG-Lipid, respectively, a result of analyzing an intensity profile on green fluorescence at a red line of the microscopy images, and a mean fluorescence intensity (MFI,×10⁴) detected by flow cytometry.

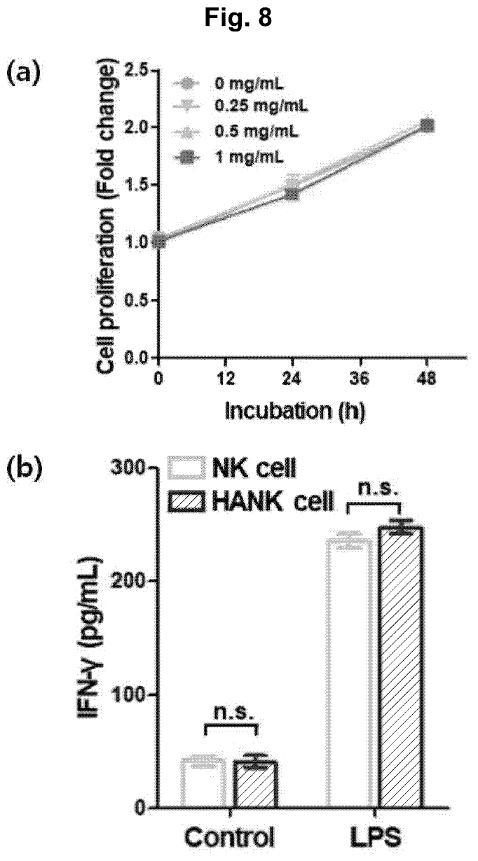

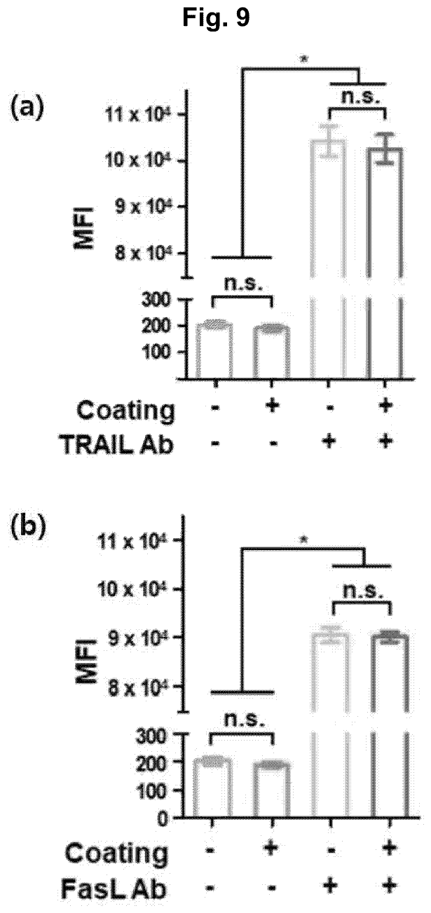

As can be seen in the fluorescence microscope image of FIG. 7, it can be confirmed that AF-HA-SH is internalized into NK cells, whereas AF-HA-PEG-Lipid is uniformly bound to the surface of the NK cells. In addition, as can be seen in the mean fluorescence intensity detected by flow cytometry, it could be confirmed that in the case of HANK cell bound to AF-HA-PEG-Lipid, an MFI value is saturated at a concentration of 1 mg/mL, whereas in the case of HANK cell bound to AF-HA-SH, a low MFI of 2.5 or less is exhibited even at a concentration of 2.5 mg/mL. In other words, it could be confirmed that HA-SH is not immobilized to the surface of the NK cell without a moiety recognizing the NK cell, whereas HA-PEG-Lipid is easily bound to the surface of the NK killer cell by lipid. FIGS. 8 and 9 are views for describing an effect of HA-PEG-Lipid on NK cells.

Referring to (a) of FIG. 8, the survival rate and proliferation rate of HANK cells in which 0 to 1 mg/mL of HA-PEG-Lipid is bound to NK cells are shown. As can be seen in (a) of FIG. 8, it can be confirmed that the survival rate and the proliferation rate are maintained in a substantially constant way despite the binding of HA-PEG-Lipid to NK cells.

Referring to (b) of FIG. 8, the result of applying LPS to HANK cell as a proinflammatory antigen signal is shown. LPS may bind to TLR4 of immune cells and induce a secretion of inflammatory cytokines such as IFN-Υ. As can be seen in (b) of FIG. 8, it could be confirmed that both NK cell and HANK cell treated with LPS exhibit a similar level of IFN-Υ secretion. In other words, it can be seen in (a) and (b) of FIG. 8 that HA-PEG-Lipid bound to the surface of the NK cell does not interfere with antigen recognition and subsequent cytokine secretion processes after intracellular signal transduction.

(a) of FIG. 9 may show the results of MFI analysis by flow cytometry after detecting TRAIL present on the surface of HANK cell using APC-bound TRAIL antibody, and (b) of FIG may show the results of making an MFI analysis by flow cytometry after detecting FasL present on the surface of HANK cell using APC-bound FasL antibody.

FasL and TRAIL may be major surface ligands of NK cells which may recognize target cancer cells and induce direct killing, and as can be seen in (a) and (b) of FIG. 9, it could be confirmed that HANK cell exhibits an MFI similar to that of antibody-treated NK cell. In other words, it can be seen that HA-PEG-Lipid does not interfere with the availability of ligands on the surface of NK cells.

### Experimental Example 2: In vitro Anticancer Effect Mediated by Immune Synapse

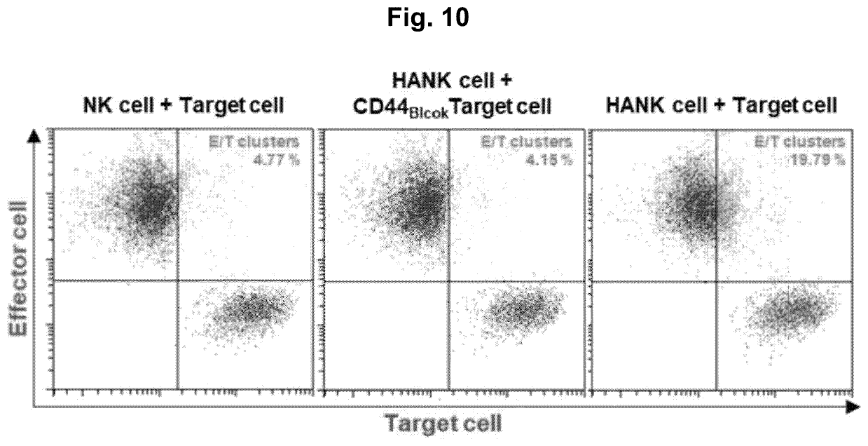

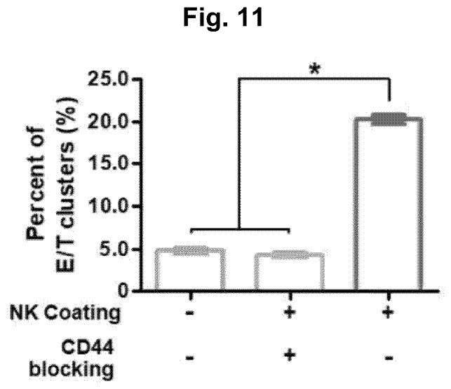

FIGS. 10 and 11 are views for confirming a target recognition ability of a HANK cell.

Referring to FIGS. 10 and 11, the target recognition ability of NK cell for target cell (NK cell+Target cell), the target recognition ability of HANK cell for CD44_{Block} target cell (HANK cell+CD44_{Block} Target cell), and the target recognition ability of HANK cell for target cell (HANK cell+Target cell) are measured and shown. Specifically, CD44 positive cancer cell lines (MIA PaCa-2, MDA-MB-231, and HCT-116) were used as a target cell, and cells in which the above-described target cell was pre-treated with 1 mg/mL of hyaluronic acid (HA) for two hours to block CD44 were used as the CD44_{Block} target cell. In addition, the target recognition ability was assessed by quantification of effector/target (E/T) cluster.

As can be seen in FIGS. 10 and 11, it could be confirmed that the target recognition ability of NK cell for target cell (NK cell+Target cell) and the target recognition ability of HANK cell for CD44_{Block} Target cell (HANK cell+CD44_{Block} Target cell) are 4.77% and 4.15%, respectively, whereas the target recognition ability of HANK cell for target cell (HANK cell+Target cell) is 19.79%, which is significantly high. In other words, it can be seen that HANK cell has a high target recognition ability for CD44.

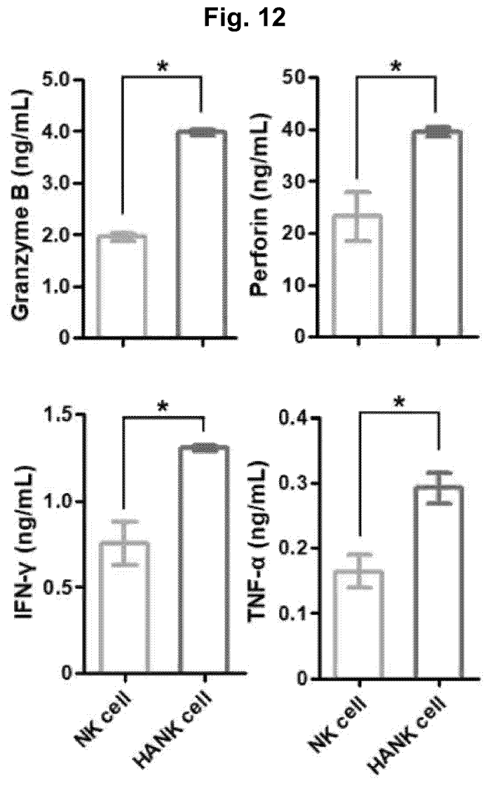

FIG. 12 is a graph comparing an amount of cytotoxic granules and cytokines secreted between NK cell and HANK cell for target cells.

Referring to FIG. 12, after co-incubating the target cell and the NK cell, an amount of secretion of cytotoxic granules (granzyme B, perforin) and cytokines (IFN-Υ, TNF-α) released from the NK cell are measured and shown. In addition, after co-incubating the target cell and the HANK cell, an amount of secretion of cytotoxic granules (granzyme B, perforin) and cytokines (IFN-Υ, TNF-α) released from the HANK cell are measured and shown. MIA PaCa-2 was used as the target cell.

As can be seen in FIG. 12, it can be confirmed that HANCK cell leads to an active secretion of cytotoxic granules (granzyme B, perforin) and cytokines (IFN-Υ, TNF-α) compared to NK cell. In other words, it can be seen that the secretion of cytotoxic granules (granzyme B, perforin) and cytokines (IFN-Υ, TNF-α) from NK cell is promoted by HA-PEG-Lipid.

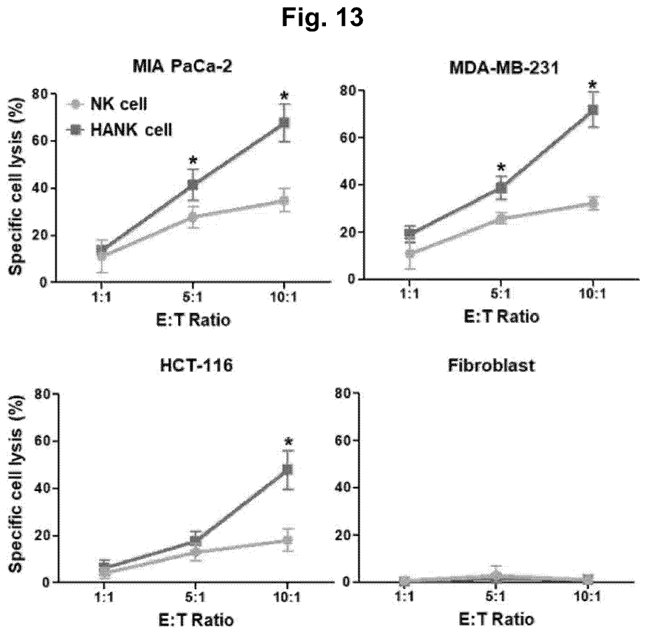

FIG. 13 is a graph comparing a lysis ability between NK cell and HANK cell for cancer cells.

Referring to FIG. 13, the NK cell and HANK cell are incubated with cancer cells (MIA PaCa-2, MDA-MB-231, HCT-116, and fibroblast), respectively, and then a cancer cell lysis rate (specific cell lysis, %) is measured and shown. An E:T ratio shown in FIG. 13 may mean an incubation ratio of NK cell or HANK cell: cancer cell.

As can be seen in FIG. 13, it could be confirmed that HANK cell exhibits a significantly higher lysis rate compared to NK cell for MIA PaCa-2, MDA-MB-231, and HCT-116 with CD44 overexpressed thereon. In other words, it can be seen that the target recognition ability of NK cell for MIA PaCa-2, MDA-MB-231, and HCT-116 is improved by HA-PEG-Lipid. In addition, it could be confirmed that HANK cell fails to lyse fibroblasts without CD44. In other words, it can be seen that HANK cell selectively recognizes cancer cells with CD44 overexpressed thereon among various cancer cells.

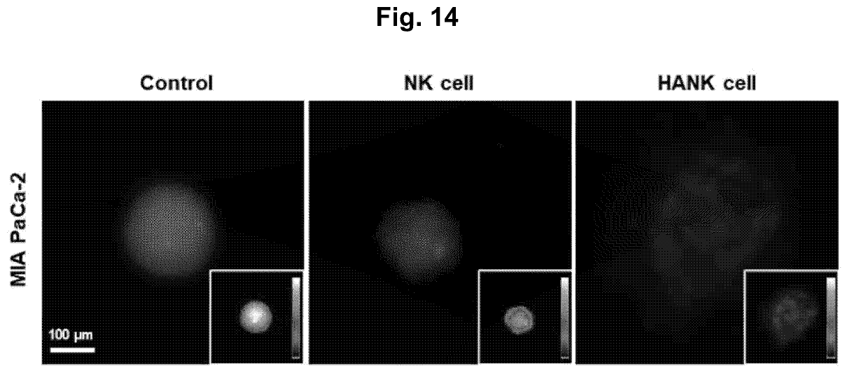

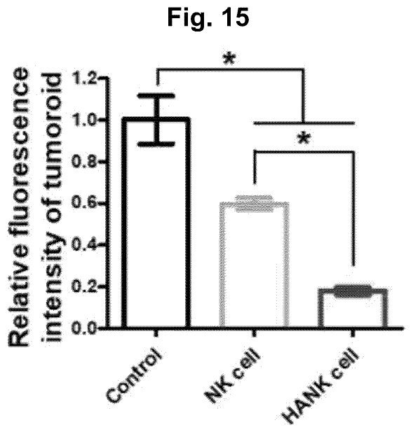

FIG. 14 and FIG. 15 are views comparing an anticancer efficacy between NK cell and HANK cell for tumor spheroids.

Referring to FIGS. 14 and 15, tumor spheroids were incubated with NK cell and HANK cell, respectively, and then an anticancer efficacy was measured by measuring a fluorescence intensity. A control shown in FIGS. 14 and 15 may mean a tumor spheroid.

As can be seen in FIG. 14, it can be confirmed that a morphology of tumor spheroids incubated with HANK cell is significantly destroyed, and as can be seen in FIG. 15, it can be confirmed that a fluorescence intensity of tumor spheroids incubated with HANK cell is significantly reduced compared to tumor spheroids incubated with NK cell.

### Experimental Example 3: Restoration of HANK Cell to NK Cell

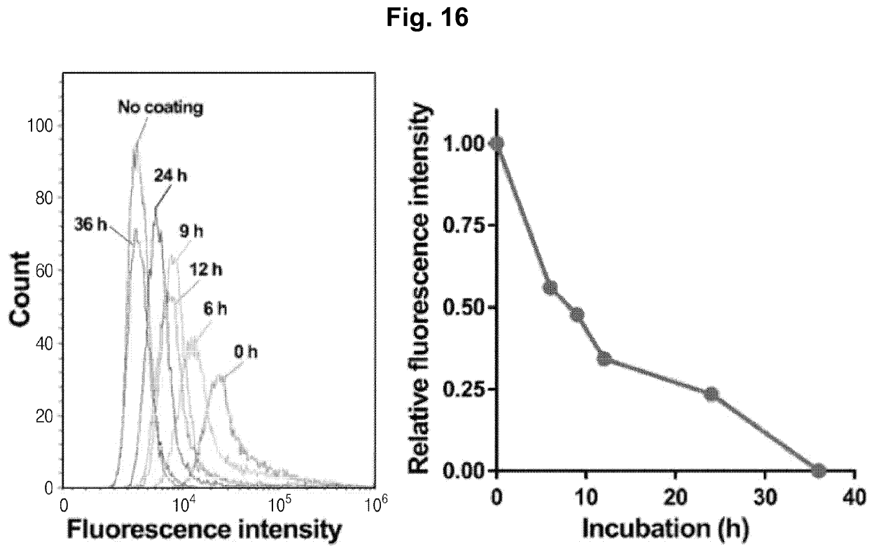

FIG. 16 is a view for confirming a retention time of HA-PEG-Lipid bound to NK cells.

Referring to FIG. 16, a retention time of HA-PEG-Lipid bound to NK cell is confirmed and shown through a fluorescence intensity analysis. As can be seen in FIG. 16, it can be confirmed that HA-PEG-Lipid bound to NK cell is removed from NK cell within 36 hours without an external physical and chemical intervention.

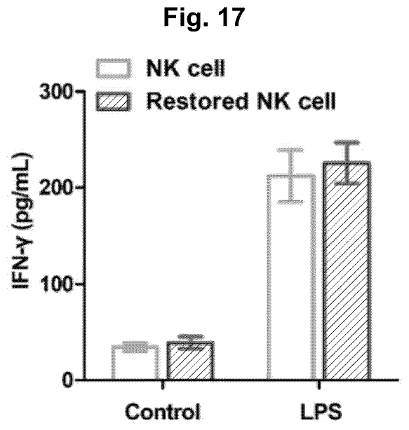

FIG. 17 is a view comparing a cytokine secretion ability between NK cell and a restored NK cell.

Referring to FIG. 17, the abilities to secrete cytokines (IFN-Υ, pg/mL) for each of NK cell and the restored NK cell are shown in comparison with each other. The restored NK cell may mean a state in which the HA-PEG-Lipid is bound to NK cell to form the HANK cell and then the HA-PEG-Lipid is removed from the NK cell. In addition, the control of FIG. 17 may show a general state, and LPS may show the result of applying LPS as an inflammatory antigen signal. LPS may bind to TLR4 of immune cells and induce a secretion of inflammatory cytokines such as IFN-Υ.

As can be seen in FIG. 17, it could be confirmed that there is no substantial difference between the NK cell and the restored NK cell with regard to the cytokine secretion ability. In other words, it can be seen that the binding and removal of HA-PEG-Lipid does not affect the cytokine secretion ability of NK cell.

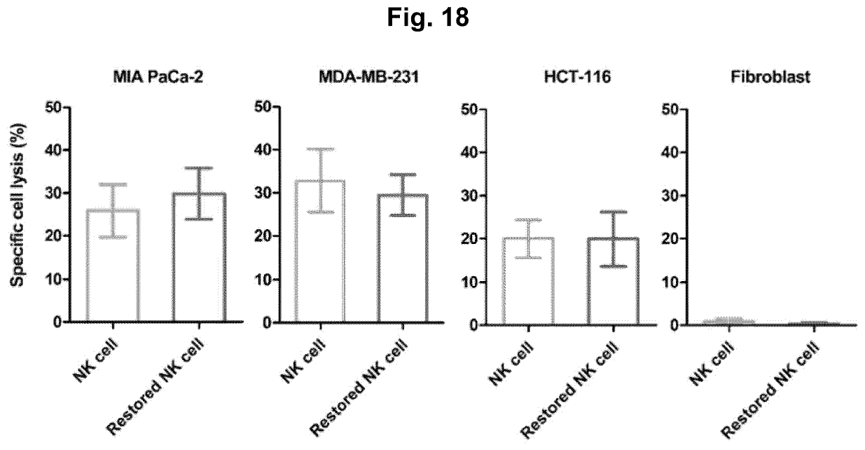

FIG. 18 is a view comparing a cancer cell lysis ability between NK cell and a restored NK cell.

Referring to FIG. 18, the abilities to lyse cancer cells for each of NK cell and restored NK cell are shown in comparison with each other MIA PaCa-2, MDA-MB-231, and HCT-116 with CD44 overexpressed thereon and fibroblast without CD44 were used as cancer cells.

As can be seen in FIG. 18, it could be confirmed that there is no substantial difference between the NK cell and the restored NK cell with regard to the ability to lyse MIA PaCa-2, MDA-MB-231, HCT-116 cancer cells In other words, it can be seen that the binding and removal of HA-PEG-Lipid does not affect the ability of NK cell to lyse cancer cells. In addition, it can be seen that the restored NK cell still does not have the ability to lyse fibroblast without CD44.

### Experimental Example 4: Evaluation of Anticancer Efficacy of HANK Cell for Pancreatic Cancer

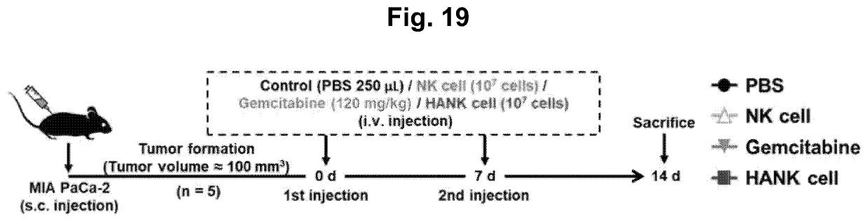

FIG. 19 is a view showing an experimental process for confirming an anticancer efficacy of HANK cell for pancreatic cancer.

Referring to FIG. 19, MIA PaCa-2, which is a pancreatic cancer cell, was injected into an experimental mouse to form a tumor, and then PBS (250 μL), NK cell (107 cell), gemcitabine (120 mg/kg), and HANK cell (107 cell) were administered thereto to evaluate an anticancer efficacy thereof, respectively. PBS was used as a control, and gemcitabine was used to confirm the efficacy of HANK cell as a drug widely known for the treatment of pancreatic cancer.

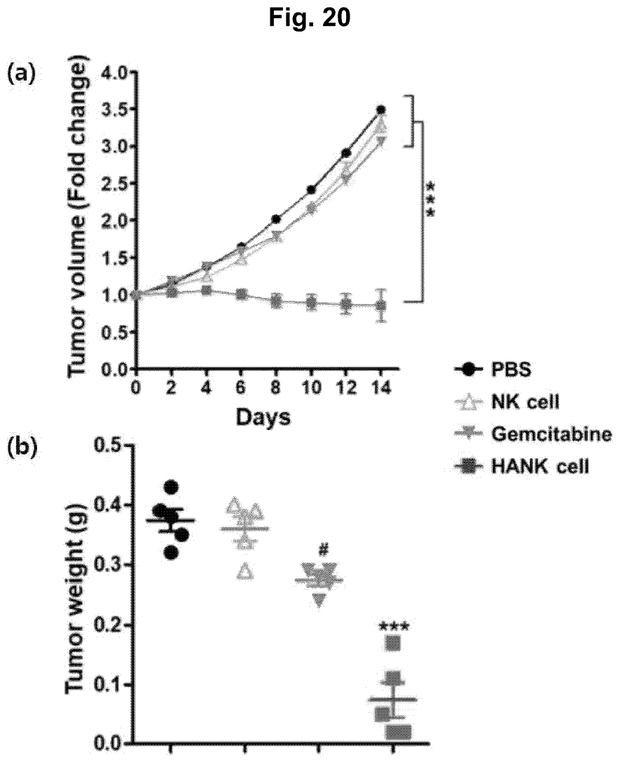

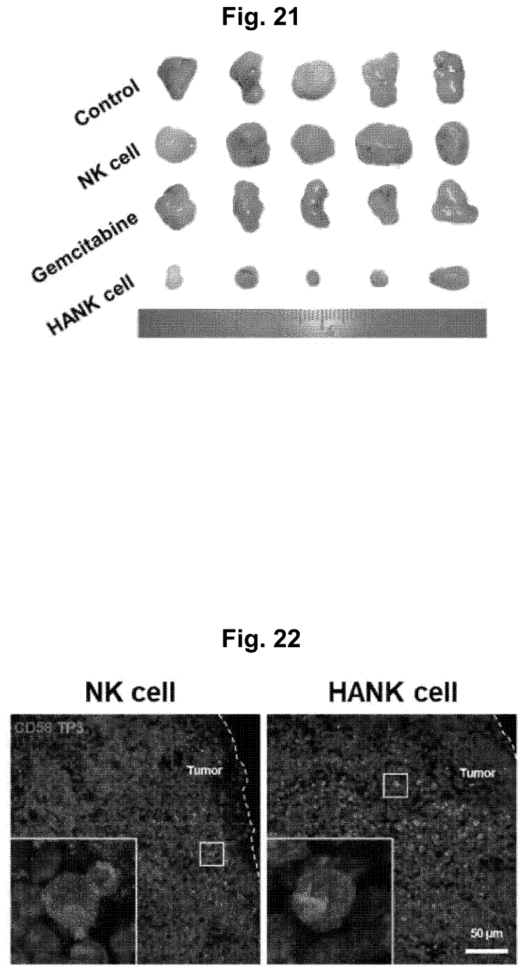

FIGS. 20 and 21 are views showing a change in volume, weight, and size of a tumor according to a progress of an experiment on pancreatic cancer.

Referring to FIGS. 20 and 21, a change in volume, weight, and size of the tumors in mice dosed with PBS, NK cell, gemcitabine, and HANK cell are shown. As can be seen in FIGS. 20 and 21, it can be confirmed that the volume, weight, and size of the tumors in mice dosed with HANK cell are significantly reduced.

FIG. 22 is a view comparing a tumor penetration ability between NK cell and HANK cell.

Referring to FIG. 22, the results of using human-specific anti-CD56 to confirm the tumor penetration ability of NK cell and HANK cell are shown. As can be seen in FIG. 22, it can be confirmed that NK cell is mainly accumulated in a boundary of a tumor, whereas HANK cell is distributed in an entire region of the tumor. In other words, it can be seen that HANK cell has a significantly higher tumor penetration ability compared to NK cell.

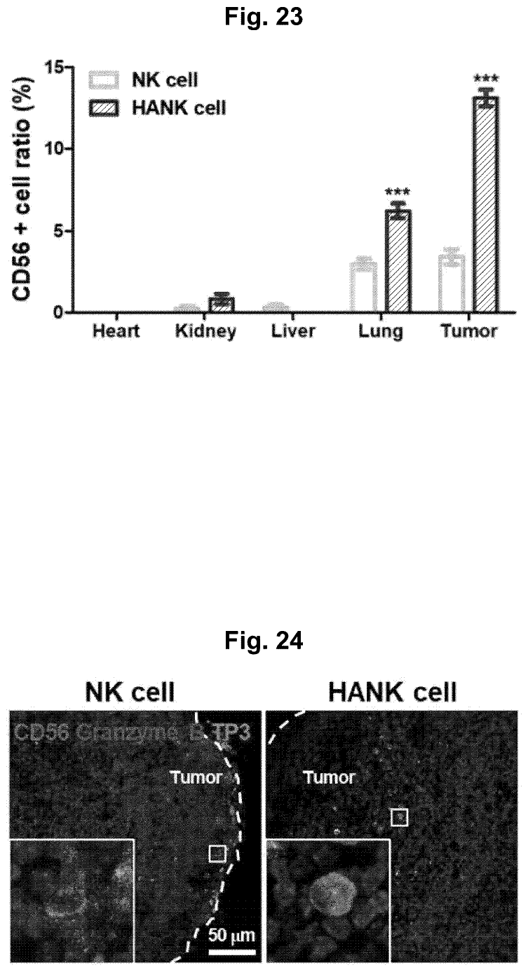

FIG. 23 is a view for confirming a biodistribution of NK cell and hank cell injected into experimental mice.

Referring to FIG. 23, the results of confirming a biodistribution after injecting NK cell and HANK cell into experimental mice are shown. Human-specific anti-CD56 was used to confirm the biodistribution.

As can be seen in FIG. 23, it could be confirmed that a very small amount of NK cell and HANK cell is detected in the heart, kidney, and liver, whereas a large amount of NK cell and HANK cell is detected in the lung and tumor. Accordingly, it can be seen that it is possible to effectively prevent pancreatic cancer from being transferred to the lung.

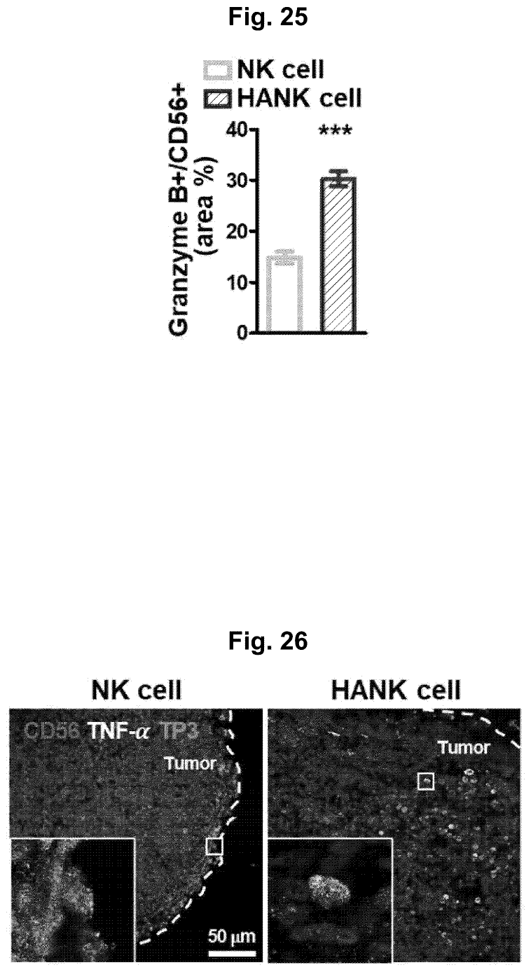

FIG. 24 and FIG. 25 are view comparing a cytotoxic granule spreading ability between NK cell and HANK cell.

Referring to FIGS. 24 and 25, the results of confirming an intratumoral distribution of granzyme B, which is a cytotoxic granule secreted from NK cell and HANK cell, are shown. Human-specific anti-CD56 was used to confirm an intratumoral distribution of granzyme B.

As can be seen in FIG. 24, it can be confirmed that granzyme B secreted from NK cell is mainly distributed in a peripheral region of the tumor, whereas granzyme B secreted from HANK cell is distributed in an entire region of the tumor. In addition, as can be seen in FIG. 25, it can be confirmed that a granzyme B+/CD56+cell area ratio (are %) is significantly higher in HANK cell compared to NK cell. Thus, it can be seen from FIGS. 24 and 25 that HA-PEG-Lipid promotes a secretion of cytotoxic granules from NK cell.

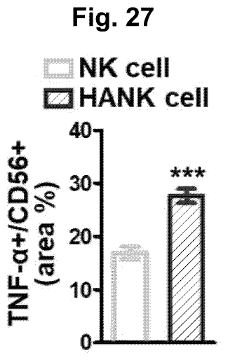

FIG. 26 and FIG. 27 are views comparing a cytokine spreading ability between NK cell and HANK cell.

Referring to FIGS. 26 and 27, the results of confirming an intratumoral distribution of TNF-α, which is a cytokine secreted from NK cell and HANK cell, are shown. Human-specific anti-CD56 was used to confirm an intratumoral distribution of TNF-α.

As can be seen in FIG. 26, it can be confirmed that TNF-α secreted from NK cell is mainly distributed in a peripheral region of a tumor, whereas granzyme B secreted from HANK cell is distributed in an entire region of the tumor. In addition, as can be seen in FIG. 27, it could be confirmed that a TNF-α/CD56+cell area ratio (are %) is significantly higher in HANK cell compared to NK cell. Thus, it can be seen from FIGS. 26 and 27 that HA-PEG-Lipid promotes a cytokine secretion from NK cell.

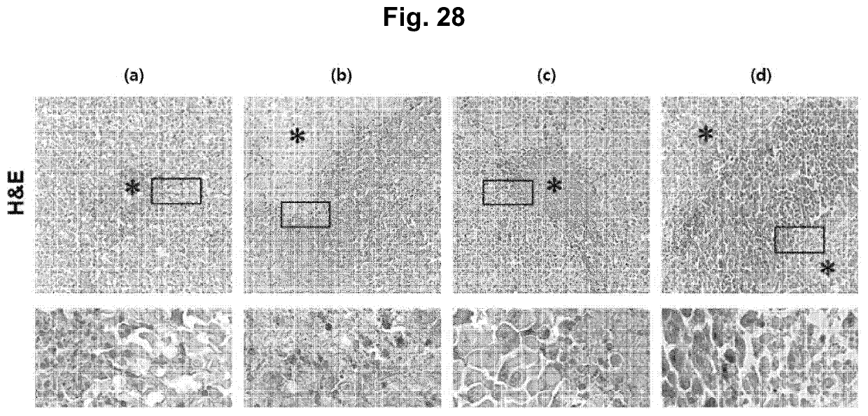

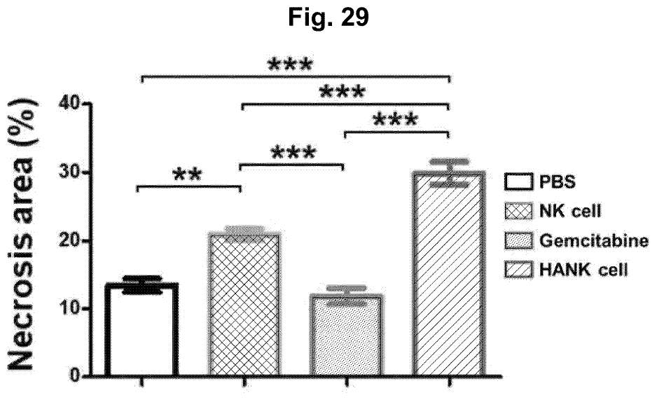

FIG. 28 and FIG. 29 are views comparing a tumor necrosis ability among PBS, NK cell, gemcitabine, and HANK cell.

Referring to FIG. 28, a degree of tumor necrosis is shown in such a way that hematoxylin and eosin (H&E) are stained on subcutaneous tumors of experimental mice injected with PBS (a), NK cell (b), gemcitabine (c), and HANK cell (d), and referring to FIG. 29, a subcutaneous tumor necrosis region (necrosis area, %) of experimental mice injected with PBS, NK cell, gemcitabine, and HANK cell is quantified and shown.

As can be seen in FIGS. 28 and 29, it can be confirmed that HANK cell exhibits a significantly improved tumor necrosis ability compared to NK cell and gemcitabine.

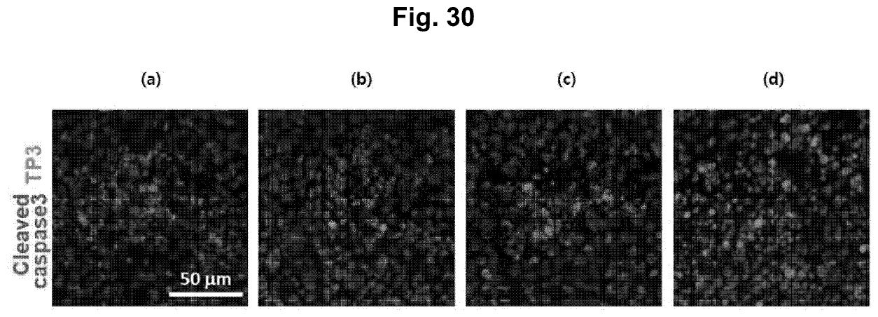

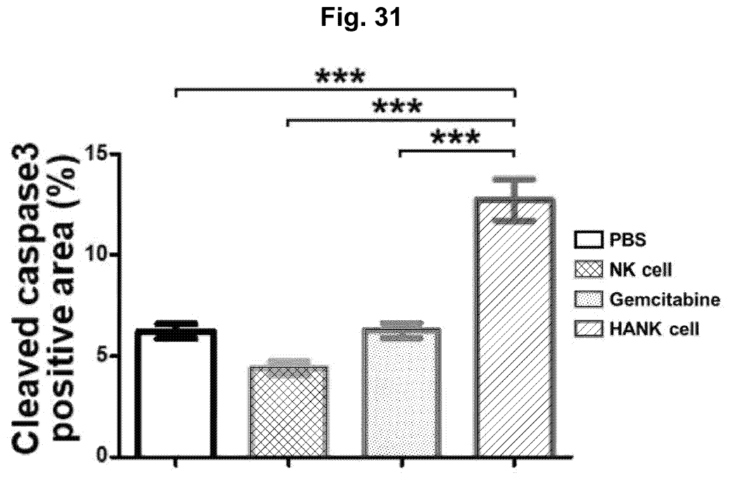

FIG. 30 and FIG. 31 are views comparing an apoptotic region of tumor masses via PBS, NK cell, gemcitabine, and HANK cell.

Referring to FIG. 30, a result of indicating an apoptotic region of a tumor mass as a cleaved caspase3 in experimental mice injected with PBS (a), NK cell (b), gemcitabine (c), and HANK cell (d) is shown, and referring to FIG. 31, a positive area of the cleaved caspase3 in experimental mice injected with PBS (a), NK cell (b), gemcitabine (c), and HANK cell (d) is quantified and shown.

As can be seen in FIG. 30, it can be confirmed that HANK cell shows a stronger expression of cleaved caspase3 compared to NK cell and gemcitabine. In addition, as can be seen in FIG. 31, it can be confirmed that HANK cell has a significantly higher positive area ratio of cleaved caspase3 compared to NK cell and gemcitabine.

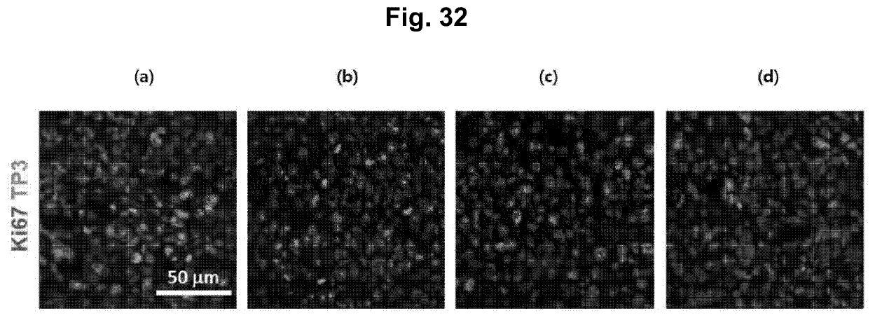

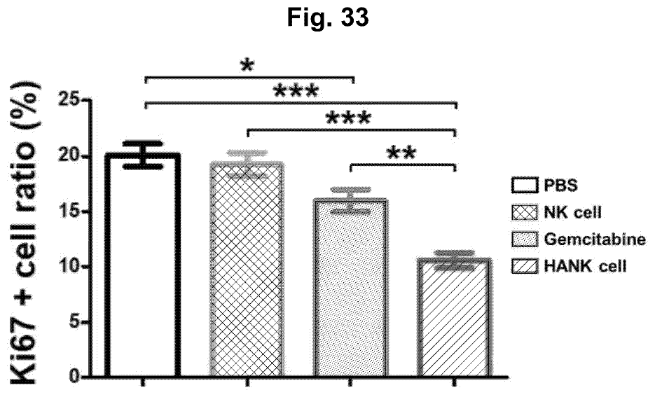

FIG. 32 and FIG. 33 are view comparing a cell proliferation inhibitory ability among PBS, NK cell, gemcitabine, and HANK cell.

Referring to FIG. 32, a result of confirming a degree of cell proliferation by staining Ki67 on subcutaneous tumors of experimental mice injected with PBS (a), NK cell (b), gemcitabine (c), and HANK cell (d) is shown, and referring to FIG. 33, a proportion of Ki67 positive cells in experimental mice injected with PBS (a), NK cell (b), gemcitabine (c), and HANK cell (d) is quantified and shown.

As can be seen in FIG. 32, it can be confirmed that HANK cell exhibits a significantly lower Ki67 expression compared to NK cell and gemcitabine. In addition, as can be seen in FIG. 33, it can be confirmed that HANK cell has a significantly lower cell proliferation region compared to NK cell and gemcitabine. In other words, it can be seen that HANK cell has a significantly higher cell proliferation inhibitory ability compared to NK cell and gemcitabine.

### Experimental Example 5: Evaluation of Antimetastatic Effect of HANK Cell

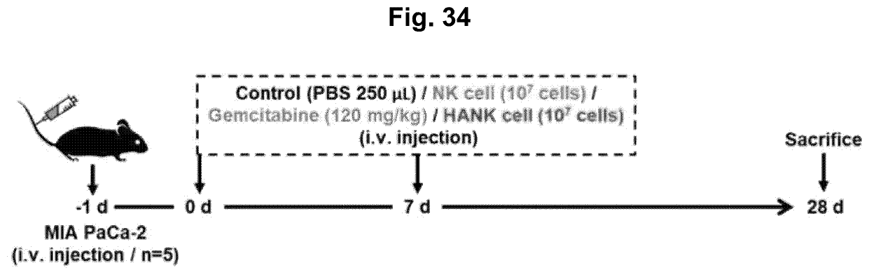

FIG. 34 is a view showing an experimental process for evaluating an antimetastatic effect of HANK cell.

Referring to FIG. 34, MIA PaCa-2, which is a pancreatic cancer cell, was injected into an experimental mouse to form a tumor, and then PBS (250 μL), NK cell (107 cell), gemcitabine (120 mg/kg), and HANK cell (107 cell) were administered thereto to evaluate an antimetastatic effect thereof, respectively. PBS was used as a control, and gemcitabine was used to confirm the efficacy of HANK cell as a drug widely known for the treatment of pancreatic cancer.

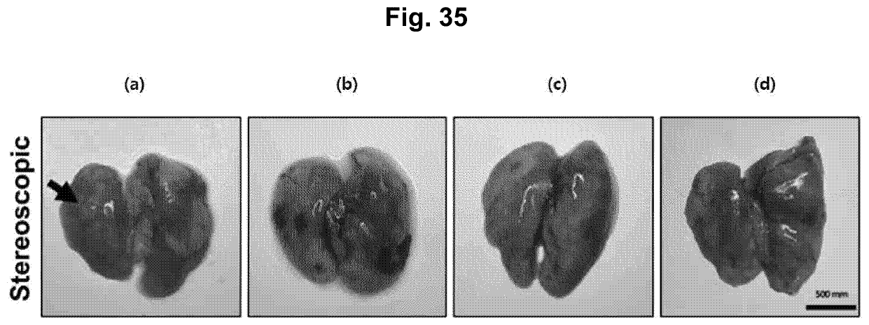

FIG. 35 is a view confirming a metastatic colony formation in lung tissues.

Referring to FIG. 35, it may show the results of confirming the formation of metastatic colonies in lung tissues of mice injected with PBS (a), NK cell (b), gemcitabine (c), and HANK cell (d). As can be seen in FIG. 35, it can be confirmed that the metastatic colony is formed in the injection of PBS (a), NK cell (b), and gemcitabine (c), but the metastatic colony is not formed

in the injection of HANK cell (d).

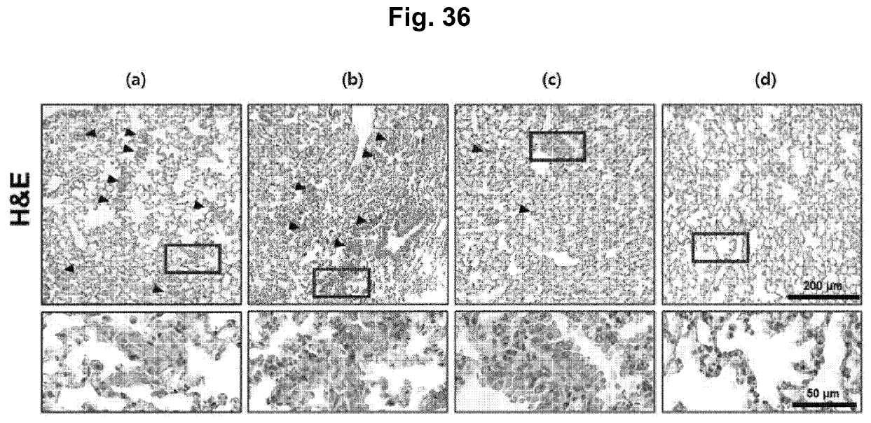

FIG. 36 is a view for confirming an alveolar structure in lung tissues.

Referring to FIG. 36, it may show the results of confirming an alveolar structure by staining the lung tissues of experimental mice injected with PBS (a), NK cell (b), gemcitabine (c), and HANK cell (d), with hematoxylin and eosin (H&E). As can be seen in FIG. 36, it can be confirmed that multiple nonalveolar cell clusters are observed when PBS (a), NK cell (b), and gemcitabine (c) are injected, whereas a typical alveolar structure is observed when HANK cell (d) is injected.

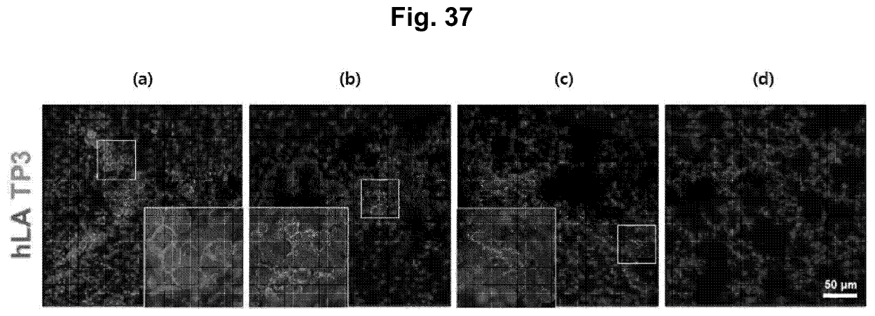

FIG. 37 is a view for describing the result of introducing a human leukocyte antigen into lung tissues.

Referring to FIG. 37, it may show the results of indicating a metastatic colony of the lung by introducing a human leukocyte antigen (hLA TP3) into the lung of experimental mice injected with PBS (a), NK cell (b), gemcitabine (c), and HANK cell (d). As can be seen in FIG. 37, it can be confirmed that an hLA positive cell cluster is detected when PBS (a), NK cell (b), and gemcitabine (c) are injected, whereas the hLA positive cell cluster is not detected when HANK cell (d) is injected.

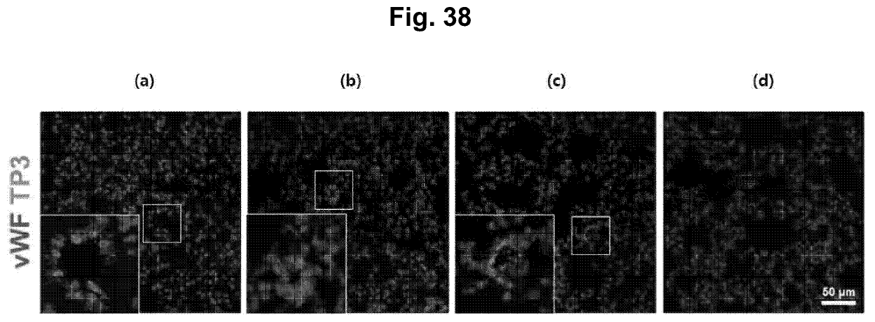

FIG. 38 is a view for describing the result of introducing an angiogenesis marker into lung tissues.

Referring to FIG. 38, it may show the results of indicating a metastatic colony of the lung by introducing von Willebrand factor (vWF) which is an angiogenesis marker of a vascularized tumor into the lung of experimental mice injected with PBS (a), NK cell (b), gemcitabine (c), and HANK cell (d). As can be seen in FIG. 38, it can be confirmed that vWF is strongly expressed when PBS (a), NK cell (b), and gemcitabine (c) are injected, whereas vWF is rarely expressed when HANK cell (d) is injected.

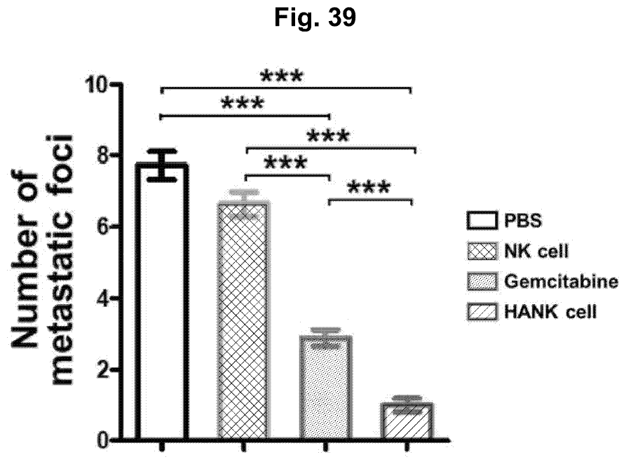

FIG. 39 is a view quantifying the number of metastatic foci in the lung identified in the lung of experimental mice injected with PBS, NK cell, gemcitabine, and HANK cell.

As can be seen in FIG. 39, it can be confirmed that the number of metastatic foci in the lung is significantly lower when HANK cell is injected compared to when PBS, NK cell, and gemcitabine are injected.

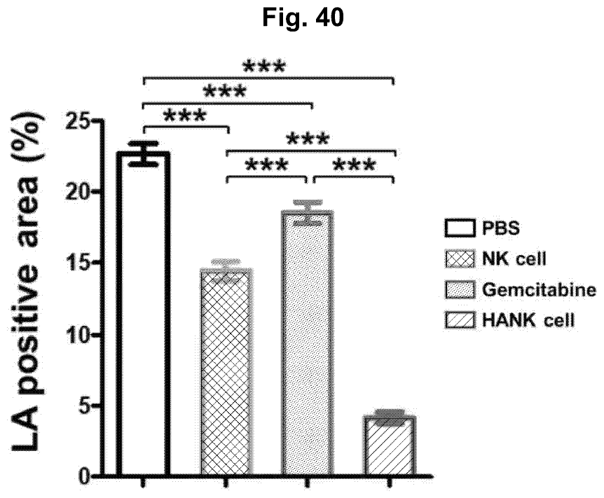

FIG. 40 is a view quantifying an hLA positive cell area identified in the lung of experimental mice injected with PBS, NK cell, gemcitabine, and HANK cell.

As can be seen in FIG. 40, it can be confirmed that the hLA positive cell area is significantly lower when HANK cell is injected compared to when PBS, NK cell, and gemcitabine are injected.

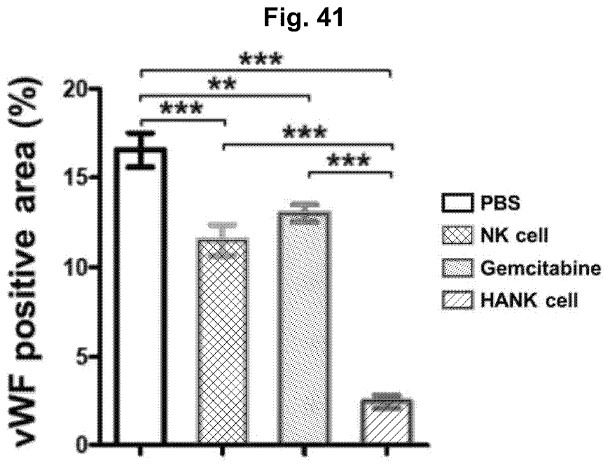

FIG. 41 is a view quantifying a vWF positive cell area identified in the lung of experimental mice injected with PBS, NK cell, gemcitabine, and HANK cell.

As can be seen in FIG. 41, it can be confirmed that the vWF positive cell area is significantly lower when HANK cell is injected compared to when PBS, NK cell, and gemcitabine are injected.

As a result, it can be seen that HANK cell in which HA-PEG-Lipid is bound to the NK cell may be easily used to treat cancer with CD44 overexpressed thereon, and may also significantly reduce metastasis to the lung.

Although the present invention has been described in detail using preferred embodiments, the scope of the present invention is not limited to specific embodiments and should be interpreted by the appended claims. In addition, it should be understood by those skilled in the art that many modifications and variations are possible without departing from the scope of the present invention.

### INDUSTRIAL APPLICABILITY

The present invention may be used in the medical industry.

1. A polymer compound comprising a hydrophobic moiety binding to a natural killer (NK) cell, a cancer cell recognition moiety, and a linker, wherein
the hydrophobic moiety is bound to one end of the linker and the cancer cell recognition moiety is bound to an other end of the linker, so as to recognize NK cells and cancer cells, in which
the cancer cell recognition moiety selectively recognizes pancreatic cancer cells, and
a cell in which the NK cell and the polymer compound are bound inhibits metastasis of pancreatic cancer cells to other organs.

2. The polymer compound of claim 1, wherein the cell in which the NK cell and the polymer compound are bound inhibits metastasis of pancreatic cancer cells to lung.

3. The polymer compound of claim 1, wherein the cancer cell recognition moiety includes hyaluronic acid.

4. The polymer compound of claim 1, wherein the polymer compound bound to the NK cell through the hydrophobic moiety is bound to the cancer cell through the cancer cell recognition moiety, and promotes a secretion of cytotoxic granules and cytokines from the NK cell to kill the cancer cell.

5. The polymer compound of claim 1, wherein the hydrophobic moiety includes a lipid, and is bound to a surface of the NK cell through a hydrophobic interaction via the lipid.

6. The polymer compound of claim 5, wherein the hydrophobic moiety includes any one of a phospholipid having an alkyl chain having 12 to 24 carbon atoms, a sterols lipid having 10 to 30 carbon atoms, 1,2-distearoyl-sn-glycero-3-phosphorylethanolamine (DSPE), 1,2-bis(diphenylphosphino)ethane (DPPE), and 1,2-bis(dimethylphosphino)ethane (DMPE).

7. The polymer compound of claim 1, wherein the linker prevents the polymer compound bound to the NK cell from being subjected to endocytosis into the NK cell.

8. The polymer compound of claim 1, wherein the linker includes polyethylene glycol (PEG).

9. The polymer compound of claim 1, wherein the polymer compound bound to the NK cell is removed from the NK cell within 36 hours.

10. The polymer compound of claim 9, wherein the polymer compound bound to the NK cell is naturally removed from the NK cell without an external physical and chemical intervention.

11. The polymer compound of claim 1, wherein the cancer cell recognition moiety recognizes solid cancer cells including CD44.

12. A method for preparing a polymer compound, the method comprising:
preparing hyaluronic acid;
thiolating the hyaluronic acid; and
reacting the thiolated hyaluronic acid with a compound in which a hydrophobic moiety is bound to one end of a linker and maleimide is bound to an other end thereof to prepare a polymer compound in which the hydrophobic moiety is bound to one end of the linker and the hyaluronic acid is bound to an other end thereof, in which
pancreatic cancer cells are recognized by the hyaluronic acid, and NK cells are recognized by the hydrophobic moiety.

13. The polymer compound of claim 12, wherein the polymer compound is prepared by a Michael reaction between the thiolated hyaluronic acid and a compound in which a hydrophobic moiety is bound to one end thereof and maleimide is bound to an other end thereof.

14. The method of claim 12, wherein the thiolating of hyaluronic acid includes:
conjugating the hyaluronic acid with 3-(2-pyridyldithithio)propionyl hydrazide (PDPH); and
adding 2-mercaptoethanol to the hyaluronic acid to which the PDPH is conjugated.
